(19) United States
(12) Patent Application Publication
(10) Pub. No.: US 2025/0161102 A1
(43) Pub. Date: May 22, 2025

(54) CONTRACEPTIVE DEVICE AND METHOD

(71) Applicant: Aaron L.J. FOX, RM Barrier Valley (CA)

(72) Inventor: Aaron L.J. FOX, RM Barrier Valley (CA)

(21) Appl. No.: 18/838,511

(22) PCT Filed: Feb. 13, 2023
(86) PCT No.: PCT/CA2023/050183

§ 371 Date: Aug. 14, 2024

Related U.S. Application Data
(60) Provisional application No. 63/309,666, filed on Feb. 14, 2022.

(51) Int. Cl. A61F 6/02; A61H 19/00; A61H 23/02

(52) U.S. Cl. CPC A61F 6/02; A61H 19/40; A61H 23/0245; A61H 2205/087

(57) ABSTRACT

A contraceptive device for a penis having an elongate body with a closed end, an inflation end, and at least one deformable wall therebetween. An interior within the elongate body is configured to hold an air pressure. An air valve extends from an exterior of the elongate body to the interior. The use of the contraceptive device comprises: inserting the elongate body into a urethra of the penis by the closed end; inserting a nozzle of a pump into the air valve; pumping air through the air valve thereby increasing the air pressure within the interior of the elongate body thereby deforming the deformable walls and forming one or more seals; and removing the nozzle from the air valve thereby sealing the interior of the elongate body.

### RELATED

The present application claims priority to PCT Applic. No. PCT/CA2023/050183, filed on Feb. 13, 2023, and U.S. Prov. Applic. No. 63/309,666, filed on Feb. 14, 2022, the contents of which are explicitly incorporated by reference in its entirety.

### FIELD

This invention is in the field of contraceptives, and more specifically to contraceptives for a penis.

### BACKGROUND

U.S. Pat. No. 4,934,999 to Bader discloses a closure device for a male urethra formed by an elongated valve device inserted therein. The valve is open at both ends and included a longitudinal through hole. The valve body includes a removable closure plug. The valve body is partly enclosed by a double-membrane tube that can be pressurized and expanded to secure the device within the urethra. The valve body is provided with at least one radial hole to permit the introduction of fluid such as air into a space between an inner and outer membrane. A hand pump is provided to supply as well as release air from the valve body.

### SUMMARY

The aspects herein in any and/or all combinations.

According to an aspect, there is provided a contraceptive device for a penis having an elongate body having a closed end, an inflation end, and one or more deformable walls therebetween. An interior within the elongate body may be configured to hold an air pressure. An air valve may extend from an exterior of the elongate body to the interior. The deformable wall(s) form one or more seals with the penis when deformed. The elongate body may be cylindrical when deflated. There may be a protrusion on the interior of the elongate body; and the protrusions surrounds an orifice of the air valve. The elongate body may be made of at least one of: a rubber, a latex, and a flexible plastic.

According to an aspect, the interior within the elongate body may vary in diameter along an axis of the elongate body to form the deformable wall(s) and one or more ribs. The rib(s) may varies in diameter around a circumference of the interior of the elongate body. The ribs may slope in an increasing diameter to the at least one deformable wall. The interior may have at least two chambers with at least one passage therebetween. The closed end may have a rounded outer surface.

According to another aspect, the deformable wall(s) may be formed of a flexible material with a stiff material between each of the at least one deformable wall.

According to another aspect, the contraceptive device may have an isolated chamber configured to house a vibrator.

According to yet another aspect, the contraceptive device may have a cap to cover the air valve.

According to another aspect, there is provided a use of the contraceptive device comprising a number of steps. The elongate body may be inserted into a urethra of the penis by the closed end. A nozzle of a pump may be inserted into the air valve. Air may be pumped through the air valve thereby increasing the air pressure within the interior of the elongate body. The nozzle may be removed from the air valve thereby sealing the interior of the elongate body. The nozzle may be reinserted into the air valve. The air pressure may be released from within the interior of the elongate body. The elongate body may be removed from the urethra of the penis.

### DESCRIPTION OF THE DRAWINGS

While the invention is claimed in the concluding portions hereof, example embodiments are provided in the accompanying detailed description which may be best understood in conjunction with the accompanying diagrams where like parts in each of the several diagrams are labeled with like numbers, and where:

FIG. 1 is a side cross-sectional view of a contraceptive device;

FIG. 2 is a side cross-sectional view of the contraceptive device according to another aspect;

FIG. 3 is a side cross-sectional view of the contraceptive device in an inflated configuration;

FIG. 4 is a side view of an inflation needle;

FIG. 5 is a side view of the contraceptive device according to another aspect;

FIG. 6 is a side view of the contraceptive device according to another aspect;

FIG. 7 is a side view of the contraceptive device according to another aspect;

FIG. 8 is a side view of the contraceptive device according to another aspect;

FIG. 9 is a perspective cross-sectional view of the contraceptive device according to another aspect;

FIG. 10 is a side cross-sectional view of the contraceptive device according to another aspect;

FIG. 11 is a block diagram of a vibrator; and

FIG. 12 is a side view of the contraceptive device according to another aspect.

### DETAILED DESCRIPTION

Illustrative embodiments of the invention are described below. The following explanation provides specific details for a thorough understanding of and enabling description for these embodiments. One skilled in the art will understand that the invention may be practiced without such details. In other instances, well-known structures and functions have not been shown or described in detail to avoid unnecessarily obscuring the description of the embodiments.

Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively. Additionally, the words “herein,” “above,” “below” and words of similar import, when used in this application, shall refer to this application as a whole and not to any particular portions of this application. When the claims use the word “or” in reference to a list of two or more items, that word covers all of the following interpretations of the word: any of the items in the list, all of the items in the list and any combination of the items in the list. When the word “each” is used to refer to an element that was previously introduced as being at least one in number, the word “each” does not necessarily imply a plurality of the elements, but can also mean a singular element.

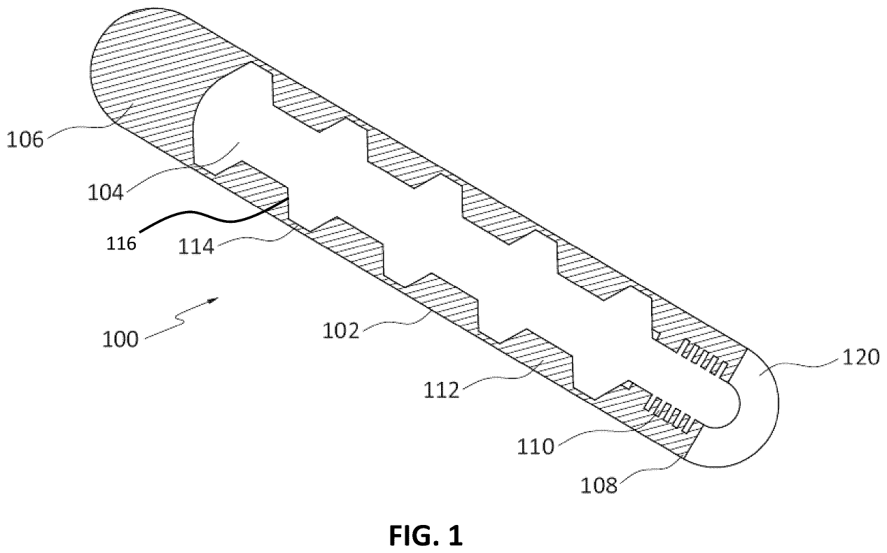

Turning to FIG. 1, a contraceptive device 100 may have an elongate body 102 and may be constructed of rubber, latex, and/or a flexible plastic. The elongate body 102 may be generally cylindrical in shape with a length of approximately 67-mm and an outer diameter of approximately 7.5-mm when deflated. Other aspects may have the length of between approximately 25-mm to approximately 31.25-mm and the outer diameter of between approximately 5-mm to approximately 7.5-mm. The elongate body 102 may have a hollow interior 104.

A closed end 106 may be at one end of the elongate body 102. The closed end 106 may have a thickness of approximately 3.5-mm from an exterior of the elongate body 102 to the hollow interior 104. The thickness of the closed end 106 may be shaped to facilitate insertion of the contraceptive device 100 into a urethra (not shown). In this aspect, the closed end 106 may have a rounded outer surface.

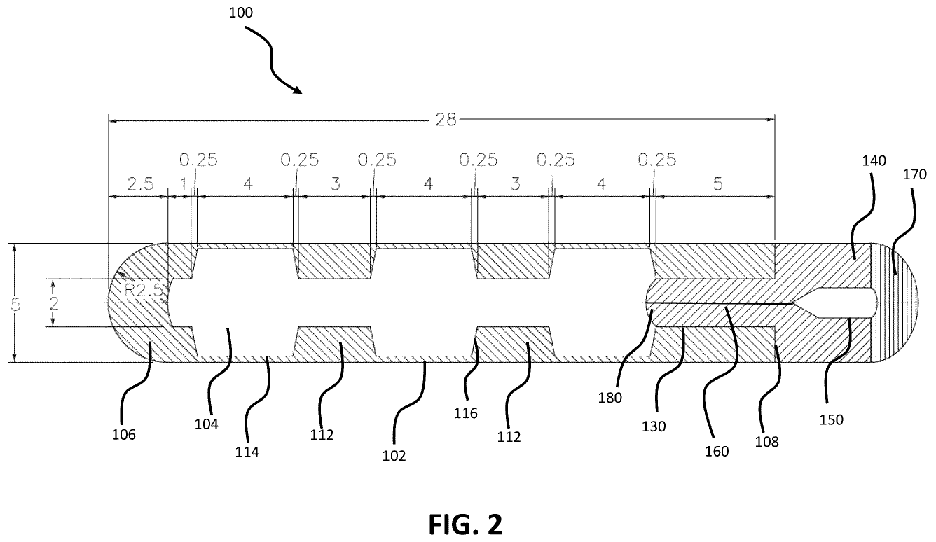

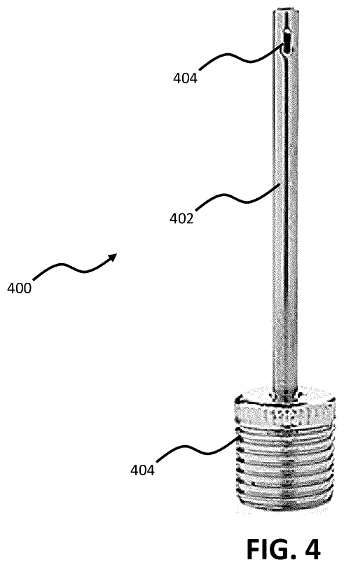

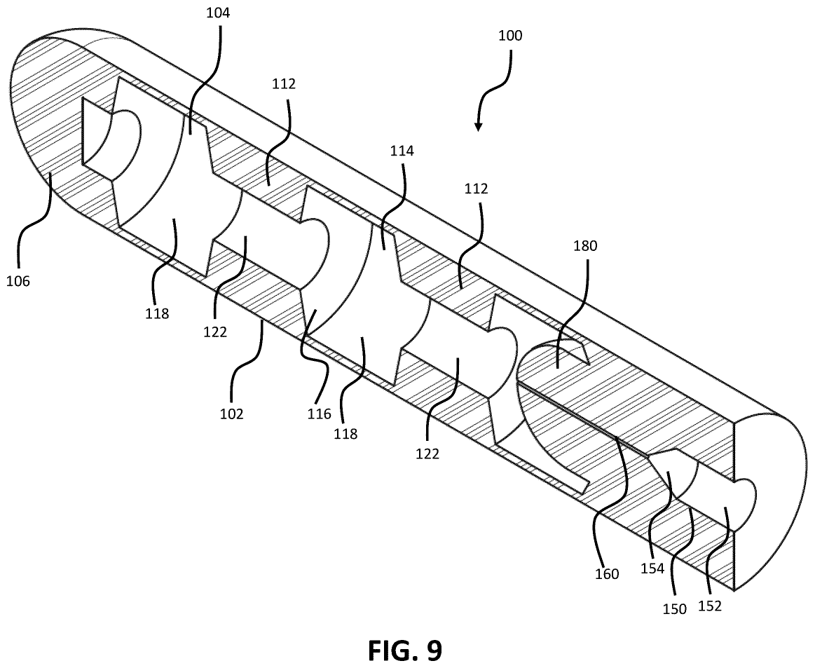

An opposite end of the elongate body 102 to the closed end 106 may be a closable end or an inflation end 108. On an interior of the closable end 108 may be threaded 110 to receive a correspondingly threaded cap 120. The threaded cap 120 on the closable end 108 may form an air-tight seal between the hollow interior 104 of the elongate body 102 and the exterior of the elongate body 102 such that the hollow interior 104 may hold pressure. In some aspects, the threaded cap 120 may be glued with adhesive or epoxy to the elongate body 102. The threaded cap 120 may comprise an air valve 160, show in further detail in FIG. 2 and FIG. 9, and similar to that disclosed in U.S. Pat. No. 4,341,382 to Arnold, herein explicitly incorporated by reference in its entirety. The air valve 160 may extend from an exterior of the elongate body 102 to the hollow interior 104. The air valve 160 may be configured to pneumatically couple to a hand pump (not shown). The hand pump may be used to inflate the elongate body 102 using an inflation needle 400 shown particularly in FIG. 4. The air valve 160 may form an airtight seal when the inflation needle 400 is not present therein. The inflation needle 400 may comprise a hollow elongate shaft 402 with an orifice 404 at one end and a coupler 404 at the other end. The coupler 404 may be threaded to be received by the hand pump. The elongate shaft 402 may be inserted into the air valve 160 until the orifice 404 enters the hollow interior 104. In some aspects, the hand pump may also have a relief valve to deflate the elongate body 102.

The hollow interior 104 may generally be cylindrical in shape and comprise one or more narrow diameter portions 112, referred to herein as ribs 112, and one or more larger diameter portions 114, referred to herein as walls 112 along an axis of the elongate body 102. An example is most clearly shown in FIG. 9. In this aspect, each of the ribs 112 may be continuous diameter around the circumference of the hollow interior 104. In other aspects, each of the ribs 112 may have a varying diameter around the circumference of the hollow interior 104 forming a plurality of ridges and valleys (not shown). The ribs 112 may slope in an increasing diameter at each end 116 (e.g. sloped end) towards the walls 112. In this aspect, the slope may be between 2.8 and 12 (e.g. (diameter of ribs—diameter of walls)/length of slope along the elongate body 102).

Shown particularly in FIG. 1, the one or more ribs 112 may be on the interior of the elongate body 102. The ribs 112 may be approximately 5.5-mm long. The ribs 112 may extend into the hollow interior 104 from the exterior of the elongate body 102 by approximately 2.25-mm. The ribs 112 may provide structural strength to the elongate body 102 and/or may prevent deformation of the elongate body 102 when under pressure. In this aspect, four ribs 112 are present. The wall 114 between each of the ribs 112 may be approximately 0.25-mm.

Turning to FIG. 2, the elongate body 102 may have a reduced length of approximately 28-mm and a reduced outer diameter of approximately 5-mm. In this aspect, the ribs 112 may extend into the hollow interior 104 from the exterior of the elongate body 102 by approximately 1.5-mm. The ribs 112 may be approximately 3-mm long with the wall 114 being approximately 4-mm between each rib 112. This aspect demonstrates two ribs 112 with a third rib 112 merging with the closed end 106 in order to provide additional strength and/or stability to the closed end 106.

The closable end 108 may comprise an enlarged cap 140 that may be friction fitted into the elongate body 102. In some aspects, the cap 140 may be secured with epoxy or adhesive. The elongate body 102 may comprise a thickened end 130 in order to provide strength for the cap 104. The thickened end 130 may be approximately 5-mm in length to receive the cap 140. The enlarged cap 140 may comprise the air valve 160. The air valve may be configured to pneumatically couple to a hand pump (not shown) via an opening or receptacle 150 for receiving the nozzle 400. The cap 140 may comprise a flippable cover 170 to close the opening 150 when not being filled.

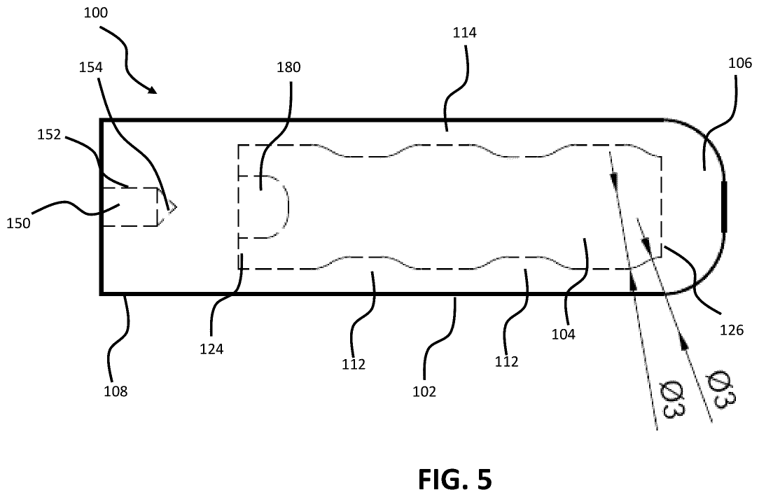

Turning to FIGS. 5-10, the contraceptive device 100 may be molded for a liquid material that forms into a flexible solid, such as rubber, latex, and/or a flexible plastic. The liquid material may be poured into a mold that forms the flat end 108. The mold may comprise an interior shape resembling the hollow interior 104. The closed end 106 may be left open at this stage to permit the interior shape to be removed from the flexible solid. Once the liquid material forms the flexible solid, the flexible solid may be removed from the mold. The open end 106 may then be placed in a rounded mold and filled with the liquid material in order to transform the open end 106 into the closed end 106 once the liquid material forms a flexible solid cap without seams. In some aspects, the closed end 106 may be polished to give the closed end 106 a smooth rounded finish.

Similar as previously described, the elongate body 102 may house the hollow interior 104. The receptacle 150 may be configured to receive the nozzle 400. In these aspects, the receptacle 150 may comprise a cylindrical portion 152 and a conical portion 154. The conical portion 154 may direct the nozzle 400 into the air valve 160. In these aspects, the receptacle 150 may be approximately 1.5-mm in diameter and/or may be approximately 3-mm in length. The air valve 160 extends from the conical portion 154 to the flat end 108 of the hollow interior 104. In order to assist with sealing the air valve 160, a protrusion 180 may encompass an orifice where the air valve 160 meets the hollow interior 104. The protrusion 180 may assist with sealing the air valve 160 and/or may provide additional material to facilitate insertion of the elongate body 102 into the penis. In some aspects, the nozzle 400 may pass into the channel for the air valve 160 through the protrusion 180. The hollow interior 104 may have a flat end 124 located near the closable end 108 and/or a tapered flat end 126 near the closed end 106. In this aspect, the closable end 108 may not have a cover 170. In other aspects, the flat end 108 may receive a plug (not shown) in the receptacle 150.

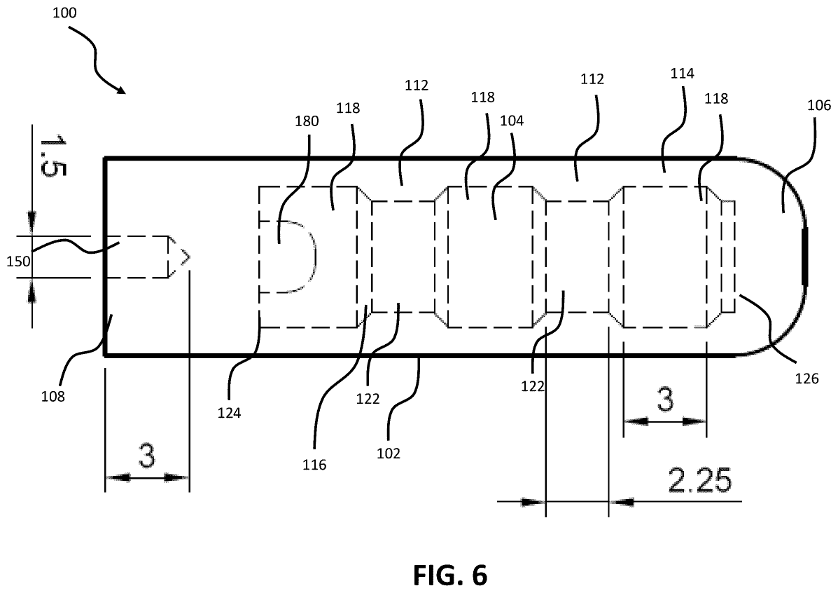

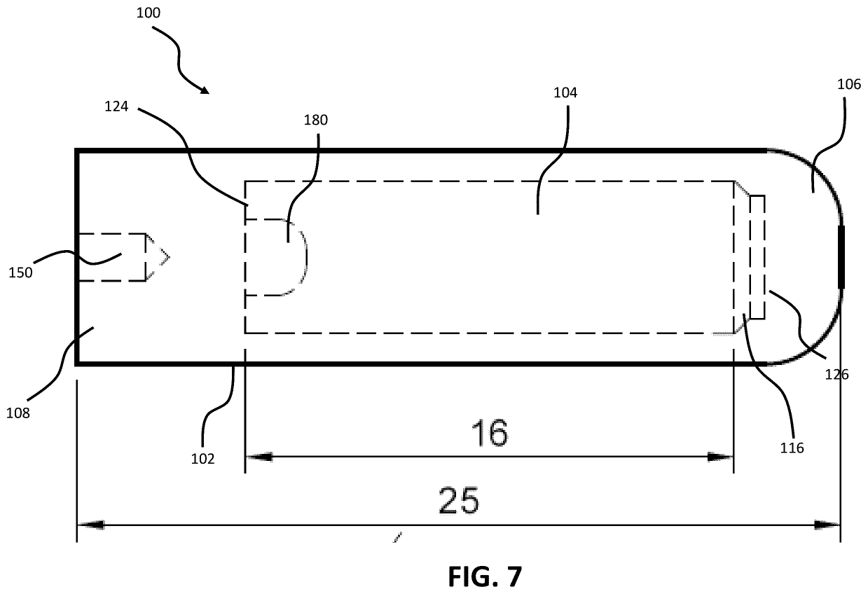

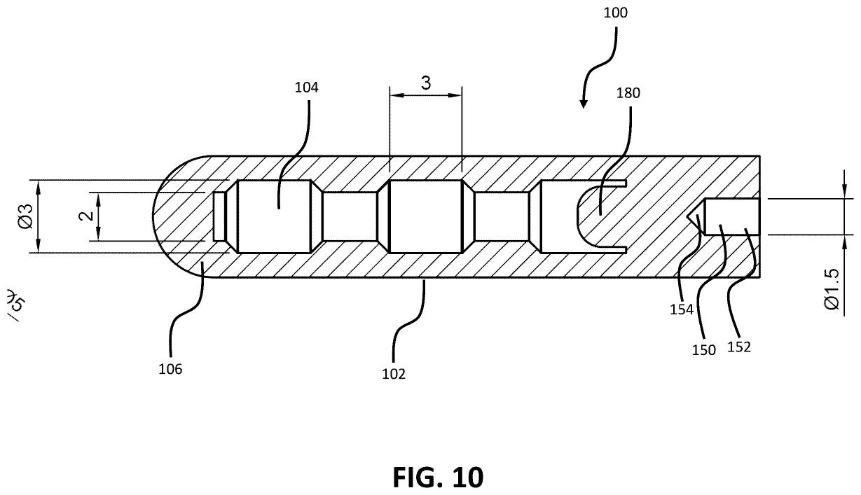

The ribs 112 and walls 114 of the hollow interior 104 may provide a number of different configurations. For example, in FIG. 5, the ribs 112 and walls 114 may be formed of one or more waves on the hollow interior 104. As shown in FIGS. 6 and 10, the ribs 112 and walls 114 may comprise a plurality of chambers 118 with passages 122 therebetween. As shown in FIG. 7, the hollow interior 104 may be a hollow cylinder with a constant diameter.

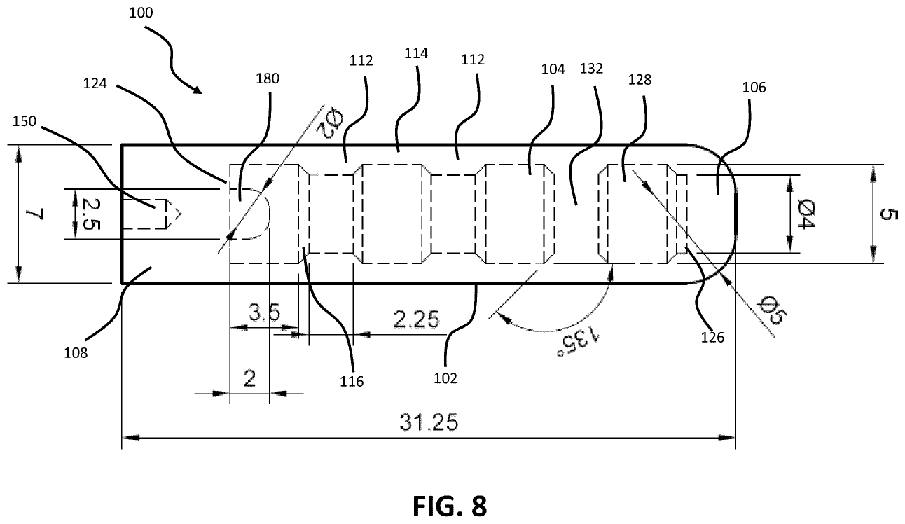

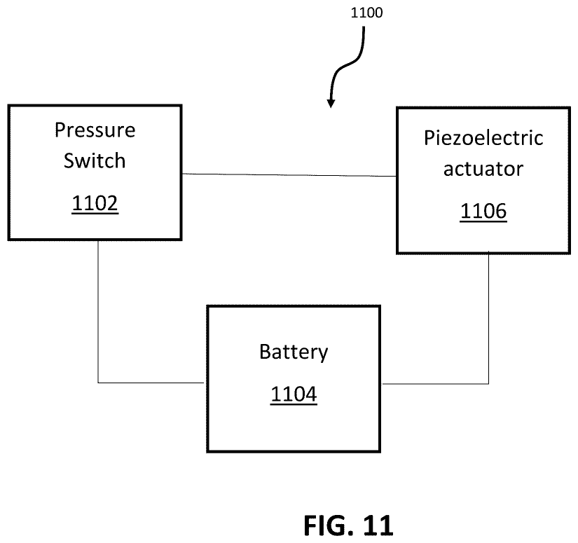

Turning to FIG. 8, the elongate member body 102 may comprise one or more isolated chambers 128. The chamber 128 may be sealed from the hollow interior 104 by solid disc of material 132 between the chamber 128 and the hollow interior 104. The chamber 128 may receive a vibrator 1100, shown in FIG. 11. The vibrator 1100 may comprise a piezoelectric actuator 1106 or vibrating motor (e.g. vibrator) that may be activated by a pressure switch 1102 such that when the hollow interior 104 is under pressure, the vibrator 1106 is provided with power from a battery 1104. In some aspects, the chamber 128 may be provided with the vibrator 1100 during manufacture by placing the vibrator into the mold prior to pouring the liquid material therein.

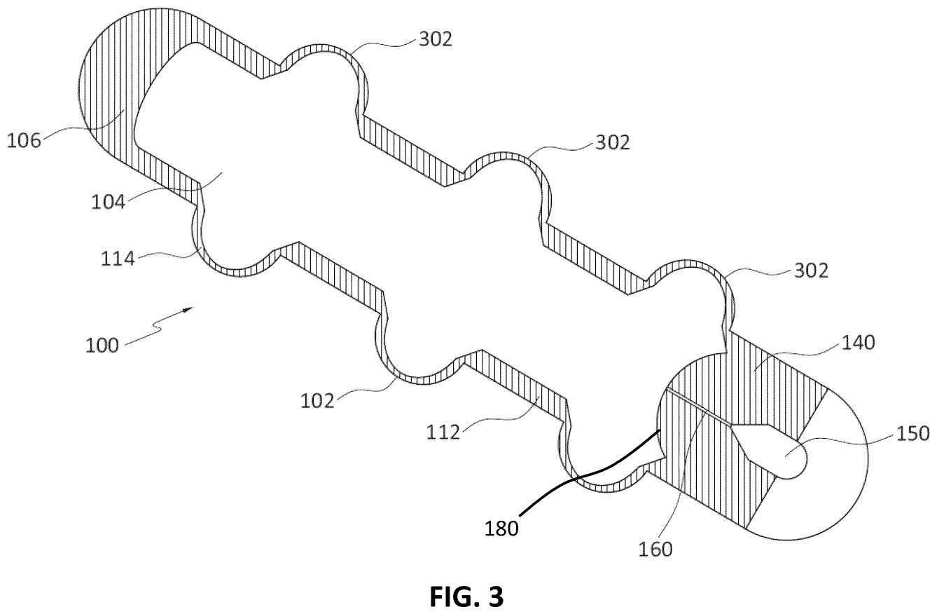

In use, the contraceptive device 100 may be inserted into a urethra of an erect penis using the closed end 106 of the elongate body 102. The cap 120, 140 may be visible at the tip of the penis. A nozzle 400 of the hand pump may be inserted into the air valve 160 within the cap 140. The hand pump may increase the pressure within the elongate body 102 causing the walls 114 between each of the ribs 112 to deform outward from a central axis of the elongate body 102 as shown particularly in FIG. 3. The deformation may be up to about 10-mm to about 20-mm. The deformation of at or near the deformable walls 114 between each of the ribs 112 may secure the elongate member 102 within the penis thereby forming a plurality of seals 302 between the elongate member 102 and the urethra. Any semen that may escape by one of the seals 302 may be contained between pairs of seals 302. As the plurality of seals 302 form, a length of the elongate member 102 may be reduced pulling the closable end 108 into the penis. The length of the elongate member 102 may reduce by between about 1.5-mm to about 2-mm per seal 302. For example, for three seals 302, the length may reduce by between about 4.5-mm to about 6-mm.

Once the use of the contraceptive device 100 is complete, the nozzle 400 of the hand pump may be placed into the valve 160 and the relief valve on the hand pump may be used to relieve the pressure. In another aspect, only the nozzle 400 may be inserted resulting in the pressurized interior 104 to be vented to atmosphere. Once the pressure is relieved, the length of the elongate member 102 may extend such that the closable end 108 may be extend out of the penis. The contraceptive device 100 may then be removed from the urethra.

Although the aspects described herein describe the contraceptive device 100 being formed of two pieces (e.g. the elongate body 102 and the cap 120, 140), other aspects may have the contraceptive device 100 formed in one complete mold. The closed end 106 may remain open during an injection of the rubber, latex, and/or a flexible plastic into a mold to form the cap 140, 160 and/or a portion of the elongate body 102 as one piece. Once formed, the internal molding may be removed, and closed end 106 may be formed. Once the closed end 106 is formed, an incision may be cut in the center of the valve end 120, 140 to allow the air needle to be inserted in order to inflate the contraceptive device 100.

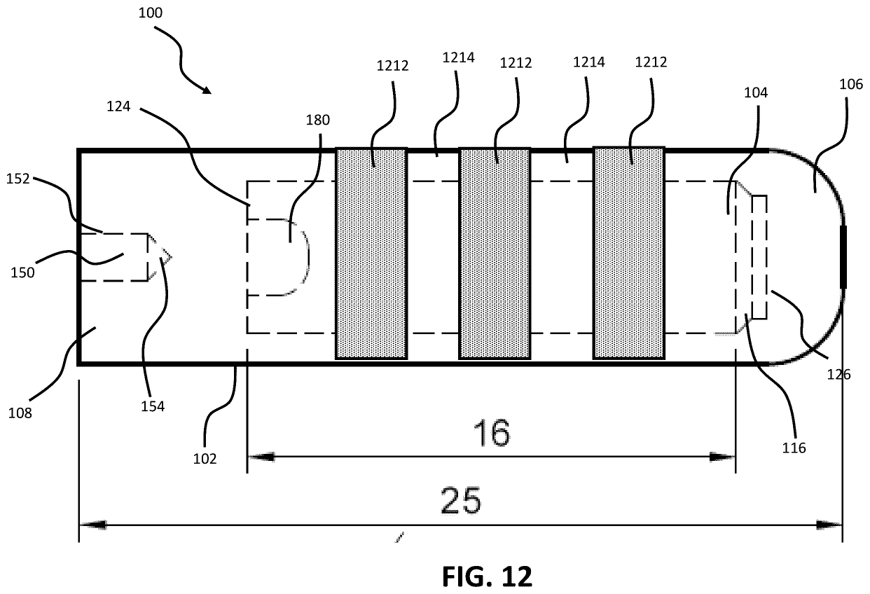

According to another aspect shown in FIG. 12, the contraceptive device 100 may comprise a dual compound mixture of a stiff material 1212 that constrains expansion and a softer (or flexible) material 1214 that allows for expansion. The stiff material 1212 may alternate with the flexible material 1214 along the elongate body 102. The stiff materials 1212 and softer materials 1214 could be selected from one or more different grades of rubber, silicone, and/or latex.

The above detailed description of the aspects of the invention is not intended to be exhaustive or to limit the invention to the precise form disclosed above or to the particular field of usage mentioned in this disclosure. While specific aspects of, and examples for, the invention are described above for illustrative purposes, various equivalent modifications are possible within the scope of the invention, as those skilled in the relevant art will recognize. Also, the teachings of the invention provided herein can be applied to other systems, not necessarily the system described above. The elements and acts of the various aspects described above can be combined to provide further aspects.

All of the above patents and applications and other references, including any that may be listed in accompanying filing papers, are incorporated herein by reference. Aspects of the invention can be modified, if necessary, to employ the systems, functions, and concepts of the various references described above to provide yet further embodiments of the invention.

Changes can be made to the invention in light of the above “Detailed Description.” While the above description details certain embodiments of the invention and describes the best mode contemplated, no matter how detailed the above appears in text, the invention can be practiced in many ways. Therefore, implementation details may vary considerably while still being encompassed by the invention disclosed herein. As noted above, particular terminology used when describing certain features or aspects of the invention should not be taken to imply that the terminology is being redefined herein to be restricted to any specific characteristics, features, or aspects of the invention with which that terminology is associated.

While certain aspects of the invention are presented below in certain claim forms, the inventor contemplates the various aspects of the invention in any number of claim forms. Accordingly, the inventor reserves the right to add additional claims after filing the application to pursue such additional claim forms for other aspects of the invention.

The foregoing is considered as illustrative only of the principles of the invention. Further, since numerous changes and modifications will readily occur to those skilled in the art, it is not desired to limit the invention to the exact construction and operation shown and described, and accordingly, all such suitable changes or modifications in structure or operation which may be resorted to are intended to fall within the scope of the claimed invention.

1. A contraceptive device for a penis comprising:
an elongate body having a closed end, an inflation end, and at least one deformable wall therebetween;
an interior within the elongate body is configured to hold an air pressure; and
an air valve extending from an exterior of the elongate body to the interior.

2. The contraceptive device according to claim 1, wherein the at least one deformable wall forms at least one seal with the penis when deformed.

3. The contraceptive device according to claim 1, wherein the elongate body is cylindrical when deflated.

4. The contraceptive device according to claim 3, wherein the interior within the elongate body varies in diameter along an axis of the elongate body to form the at least one deformable wall and at least one rib.

5. The contraceptive device according to claim 4, wherein the at least one rib varies in diameter around a circumference of the interior of the elongate body.

6. The contraceptive device according to claim 3, wherein the at least one rib slopes in an increasing diameter to the at least one deformable wall.

7. The contraceptive device according to claim 1, further comprises a protrusion on the interior of the elongate body; and the protrusion surrounds an orifice of the air valve.

8. The contraceptive device according to claim 1, wherein the at least one deformable wall is formed of a flexible material with a stiff material between each of the at least one deformable wall.

9. The contraceptive device according to claim 1, further comprising an isolated chamber configured to house a vibrator.

10. The contraceptive device according to claim 1, further comprising a cap to cover the air valve.

11. The contraceptive device according to claim 3, wherein the interior comprises at least two chambers with at least one passage therebetween.

12. The contraceptive device according to claim 1, wherein the closed end has a rounded outer surface.

13. The contraceptive device according to claim 1, wherein the elongate body comprises at least one of: a rubber, a latex, and a flexible plastic.

14. A use of the contraceptive device according to claim 1, comprises:
inserting the elongate body into a urethra of the penis by the closed end;
inserting a nozzle of a pump into the air valve;
pumping air through the air valve thereby increasing the air pressure within the interior of the elongate body; and
removing the nozzle from the air valve thereby sealing the interior of the elongate body.

15. The use according to claim 14, further comprises:
reinserting the nozzle into the air valve;
releasing the air pressure from within the interior of the elongate body; and
removing the elongate body from the urethra of the penis.
